(19) United States
(12) Patent Application Publication
(10) Pub. No.: US 2012/0046314 A1
(43) Pub. Date: Feb. 23, 2012

(54) NITROXIDES FOR USE IN TREATING OR PREVENTING NEOPLASTIC DISEASE

(75) Inventors: Louis Habash, Newport Beach, CA (US); Clarence Jones, Huntington Beach, CA (US)

(73) Assignee: MITOS, INC., Newport Beach, CA (US)

(21) Appl. No.: 11/815,444

(22) PCT Filed: Feb. 2, 2006
(86) PCT No.: PCT/US06/03972

§ 371 Date: Aug. 22, 2008

Related U.S. Application Data
(60) Provisional application No. 60/649,230, filed on Feb. 2, 2005.

(51) Int. Cl. A61K 31/445; C07D 211/94; A61P 35/00

(52) U.S. Cl. 514/315; 546/242

(57) ABSTRACT

Pharmaceutical compositions are provided that are useful in treating or preventing neoplastic disease, such as cancer. The compositions comprise a pharmaceutically acceptable carrier, and an effective therapeutic or prophylactic amount of a nitroxide antioxidant that alters the expression of one or more genes related to the cancer. Methods are also provided for the use of the pharmaceutical compositions in the treatment or prevention of cancer. In a preferred embodiment, the nitroxide antioxidant is Tempol (4-hydroxy-2,2,6,6-tetramethylpiperidine-1-oxyl), and the cancer is esophageal cancer, hepatocellular carcinoma, colon cancer, prostate cancer, lung cancer, gastric carcinoma, renal cell carcinoma, bone cancer, breast cancer, cervical cancer, brain cancer, or a cancer associated with the tumor suppressor gene p53.

### BACKGROUND OF THE INVENTION

1. Field of the Invention

The present invention relates to pharmaceutical compositions useful for treating or preventing neoplastic disease, including various cancers, and to methods for using these compositions in treating or preventing these conditions.

2. Description of the Related Art

### 1. Cancer and Oxidative Stress

Oxidative stress, the generation of free radicals within cells, is the result of a number of cellular processes. Ionizing radiation, for example, can interact with water molecules to generate hydroxyl and other radicals. Radicals may also be generated by the metabolism of oxygen via mitochondrial electron transport chains, which has the potential to donate electrons to oxygen, resulting in the formation of superoxides. Other radicals formed by these processes include hydrogen peroxide and peroxynitrite.

Cells have a number of defense mechanisms to counteract these radicals. There are a number of endogenous antioxidants that scavenge radicals, such as glutathione, ubiquinol, bilirubin, uric acid, and albumin. There are also enzymes, such as superoxide dismutase and catalase, that inactivate radicals. Ions of transition metals, which can catalyze the formation of hydroxyl radicals, are eliminated by metallothioneins, ferritin, transferrin and ceruloplasmin. However, these defense mechanisms are not able to eliminate all cellular radicals.

The radicals that escape cellular defense mechanisms can cause damage to cell structures. In particular, radicals can affect DNA, by inducing modifications of nucleotides and causing oxidation of proteins and lipid peroxidation. Base modifications, in particular, are linked to cancer: 8-hydroxyguanine, the most common lesion found after irradiation of DNA, has been reported to be a key biomarker related to carcinogenesis. (Floyd R A, Carcinogenesis 11:1447-1450, 1990) Additionally, radicals can interact with the sugar-phosphate backbone of the DNA molecule, leaving single- or double-stranded breaks that can give rise to mutations and carcinogenesis. (Karbownik et al., Proc Soc Exp Biot Med. 2000 October; 225(1):9-22)

Reduction of the deleterious activity of free radicals within cells would therefore lead to the prevention of a significant proportion of cancer and would be highly desirable.

### 2. Esophageal Carcinoma

Esophageal carcinoma arises in the mucosal layers and tends to invade the submucosa and the muscular layer, followed by invasion of nearby structures such as the tracheobronchial tree, the aorta, or the recurrent laryngeal nerve. It also spreads to the nearby lymph nodes and from there to one or both of the liver and lungs.

Approximately 14,000 new cases of the disease are diagnosed every year in the United States, and it causes approximately 11,000 deaths per year. Until the 1970s, most esophageal carcinoma was squamous cell carcinoma, and it was most often found among African-Americans with a history of smoking and alcohol consumption. The high incidence of this form of the disease makes esophageal cancer 50% more prevalent among African-Americans than among whites. A different form of the disease, adenocarcinoma, is found at higher rates among whites and is associated with gastroesophageal reflux disease and Barrett's esophagus.

Worldwide, esophageal cancer is the seventh leading cause of cancer death. Rates of incidence in Iran, northern China, India, and southern Russia are 10-100 times those in the United States. Squamous cell carcinoma is responsible for 95% of all esophageal cancer worldwide.

Esophageal cancer is usually diagnosed only at a late stage and is very difficult to cure. Because of this, it would be desirable to identify genes related to the disease and alter the expression patterns of those genes so as to prevent the development or progression of the disease from early to late stages.

### 3. Hepatocellular Carcinoma

Hepatocellular carcinoma is the fifth most common cancer and the third leading cause of cancer death in the world. This cancer is increasing in frequency in the United States, from an incidence of 1.4/100,000 in 1976-1980 to 2.4/100,000 in 1991-1995. Liver cancer often remains undiagnosed initially when it occurs in patients with underlying cirrhosis; the clinical symptoms, such as abdominal pain, are often taken to indicate progression of the underlying disease. Because of this difficulty in identifying liver cancer at an early stage, patients with resectable tumors at the time of diagnosis are rare (only 10-20% of hepatocellular carcinomas). Even patients with resectable disease exhibit only a 10-20% rate of recurrence-free survival, while few treatments are available for unresectable disease, as neither radiation therapy nor chemotherapy has proved to be of use. Alternate treatments, such as immunotherapies, have not yet been successful.

Gene therapy offers a potential alternative for the treatment or prevention of this disease. To this end, it would be desirable to identify genes related to hepatocellular carcinoma and develop methods of altering the expression patterns of those genes so as to prevent the development of the disease or slow its progression.

### 4. Colorectal Cancer

Colorectal cancer is the second leading cause of cancer-related death in the United States. In 2000, this disease caused 56,300 deaths. The disease is associated with dietary risk factors, particularly diets high in calories and animal fats. Screening programs for colorectal cancer have shown serious limitations. Simple methods, such as the Hemoccult test, fail to detect approximately half of patients with colorectal tumors, because the tumors exhibit only an intermittent bleeding pattern. Additionally, the test returns 2-4% false positive results. More invasive screening procedures, such as sigmoidoscopy, barium enema, and colonoscopy, are not only expensive and uncomfortable but also carry a risk of significant complications. Screening programs for colorectal cancer are accordingly unsatisfactory and patients often present with advanced or metastatic disease, at which point the five-year survival rate is only 5%.

The preferred method of treating colorectal cancer is the total surgical resection of the tumor. In advanced cases of the disease, however, resection is undertaken primarily to alleviate tumor-related symptoms such as gastrointestinal bleeding or obstruction. Radiation therapy is indicated in cancers of the rectum, but is of no effect in treating cancers of the colon. Chemotherapy with 5-FU is of marginal benefit in patients with advanced colorectal cancer.

For these reasons, it would be desirable to develop alternate methods of preventing or treating colorectal cancer. Gene therapy is one such alternate approach: the identification of genes related to the disease, and the development of methods for altering the expression patterns of those genes, represent a potentially promising approach to combating colorectal cancer, especially in patients with advanced disease, who benefit little from current therapies.

### 5. Lung Cancer

Primary carcinoma of the lung affects nearly 200,000 people each year in the United States, and more than 80 percent of these will die within five years of diagnosis. It is thus the leading cause of cancer death in the United States in both men and women: it accounts for 31 percent of all cancer deaths in men and 25 percent in women. At diagnosis, only approximately 15 percent of patients have a strictly local tumor, while 25 percent show spread to regional lymph nodes, and more than 55 percent have distant metastases. The five year survival rate of patients with local disease is 50 percent, while for patients with regional disease, it is 20 percent, and only 14 percent overall. Although the five year overall survival rate has nearly doubled in the last 30 years, advances in combination therapy with surgery, radiotherapy, and chemotherapy, primary carcinoma of the lung is still a major health problem with a poor prognosis.

Because lung cancer patients often present with disease that is spread beyond the initial tumor site, surgical options are often limited, and chemotherapy and radiotherapy are the only options. However, as shown by the generally poor five year survival rate, these therapies are less than optimal, and are often difficult to tolerate. Because of this, it would be desirable to identify genes related to lung cancer and to alter the expression patterns of those genes so as to prevent the development or progression of the disease from early to late stages.

### 6. Gastric Carcinoma

The instance of gastric adenocarcinoma in the United States has decreased over the course of the past 60 years, to a level of 5.0 per 100,000 in men and 2.3 per 100,000 in women. In 2000, 21,500 new cases of stomach cancer were diagnosed in the United States, and 13,000 Americans died of the disease. Although the incidents have been falling in the United States, the frequency of gastric cancer remains high in other areas, notably East Asia. Gastric carcinoma in early stages often produces no symptoms. The tumors spread by extension to the gastric wall to the perigastric tissues and adjacent organs, such as the pancreas, colon, or liver. The primary tumor frequently metastasizes, with the liver being the most common site for metastatic spread.

The only chance for a cure of gastric cancer is a complete surgical removal of the tumor, along with resection of neighboring lymph nodes. However, fewer than one third of patients present with tumors that are susceptible to such treatment. The five year survival rate for the 25 to 30 percent of patients that are able to undergo complete resection of the tumor is approximately 20 percent for distal tumors, and less than 10 percent for proximal tumors. The tumor is relatively resistant to radiotherapy, and chemotherapy generally produces only transient partial responses, with complete remissions being a rarity.

For these reasons, it would be desirable to develop alternative methods for preventing or treating gastric carcinoma. Gene therapy is one such alternate approach: the identification of genes related to the disease, and the development of methods for altering the expression patterns of the disease, represent a potentially promising approach to combating gastric carcinoma.

### 7. Renal Cell Carcinoma

Renal cell carcinoma accounts for 90 to 95 percent of kidney cancer. In the year 2000, there were 31,200 new cases of renal cancer, and 11,900 people died of the disease in the United States. The five year survival rate is 60 to 65 percent for early stage disease, while later stages have a rate of 20 percent or less. The standard treatment for early stage disease is radical nephrectomy, including adjacent lymph nodes. Survival is very poor in cases of metastatic disease; chemotherapy has not been promising, while therapy using IL-2 and alpha interferon produce responses in 10 to 20 percent of patients, but in most, the response is transient.

Because of the relatively poor treatment options, it would be desirable to develop alternate methods of treating the disease, such as gene therapy. It would, therefore, be desirable to identify genes related to renal cell carcinoma and to alter the expression patterns of those genes so as to prevent the development or progression of the disease.

### 8. Bone Sarcoma

Bone sarcomas account for a small percentage of all new malignances; there were approximately 2,500 new cases in the United States in 1999. Two of the most common malignant tumors of bone are osteosarcoma and Ewing's Sarcoma. Both of these tumors are common in childhood and adolescence. The standard management of osteosarcoma is a course of pre-operative chemotherapy followed by surgery and a second course of chemotherapy after surgery. Radiotherapy is not effective in treating osteosarcoma. Ewing's sarcoma is a more aggressive form of bone sarcoma, and frequently metastasizes to lung, other sites in the bone, and bone marrow.

Although bone sarcomas are often curable, the cure, as noted above, involves a course of chemotherapy followed by surgery. It would be desirable to develop alternative methods of preventing or treating such bone sarcomas. For example, the identification of genes related to bone sarcoma, coupled with a method of altering the expression patterns of those genes, could lead to effective gene therapy that would either prevent the development of the disease or slow its progression.

### 9. Prostate Cancer

Cancer of the prostate is the most common type of cancer in men and in the United States, it is the second leading cause of cancer death. In 2000, 180,400 cases were diagnosed and 31,900 men died of prostate cancer. Treatment methods vary based on the state of the disease; they include radical prostatectomy, radiation therapy, and hormone therapy. Surgical treatment carries with it the risk of side effects including incontinence or impotence.

In view of these complications, it would be desirable to develop alternate methods of preventing or treating prostate cancer. For example, if genes could be identified which were related to prostate cancer, and methods were available for altering the expressing patterns of these genes, then gene therapy could be employed to prevent or treat prostate cancer.

### 10. Breast Cancer

Breast cancer is a malignant proliferation of epithelial cells lining the ducts or lobules of the breast. There are approximately 180,000 cases of invasive breast cancer, and 40,000 deaths from the disease, per year in the United States. With the exception of skin cancer, malignancies of the breast are the most common cause of cancer in women and represent about one third of all cancers. Breast cancer can have a genetic component. Treatment options include lumpectomy or radical mastectomy with or without irradiation. Adjuvant chemotherapy or hormone therapy regimens are also often employed.

Because of the prevalence of breast cancer and the undesirable aspects involved in surgical treatment, it would be desirable to develop alternate methods of treating breast cancer, such as gene therapy. If genes were identified that were related to breast cancer and a method was developed for altering the expression patterns of the genes, this would represent a promising approach to combating the development or progression of the disease.

### 11. Cervical Cancer

There are approximately 13,000 new cases of invasive cervical cancer per year, and more than 50,000 cases of carcinoma in situ per year. In the year 2000, there were approximately 4,600 deaths from the disease. Worldwide, it is the major gynecologic cancer in underdeveloped countries. Treatment options include radical hysterectomy, radiation therapy, and platinum-based chemotherapy, based on the staging of the disease.

Because of the prevalence of this disease, particularly in underdeveloped countries, it would be desirable to develop gene therapies, in which the expression of genes associated with the development or progression of cervical cancer was altered in order to retard the development or progression of the disease.

### 12. Brain Tumors

Tumors of the brain occur in approximately 18,000 persons per year and account for an estimated 13,300 deaths in the United States every year. Of the tumors of the brain, glioma is the most common. Metastases to the brain from other primary tumor sites within the body are also very common. Brain tumors often present with a focal neurologic deficit, a seizure, or a non-focal neurologic problem, such as a headache or personality change. Treatment of brain tumors involves surgical resection, where possible, chemotherapy, or radio therapy.

Because of the negative aspects of such treatment options, it would be desirable to develop less invasive methods of treating these tumors. For example, it would be desirable to develop methods by which the expression patterns of genes related to the development of progression of brain tumors could be altered so as to retard the development of progression of such tumors.

13. p53-Related Cancers

The p53 protein is the most well-known of the “tumor suppressor” genes: these are genes, the inactivation of which contributes to the development of cancer. Briefly, p53 serves as a checkpoint to arrest cells with damaged DNA. Damaged DNA stabilizes fully functional p53, which is ordinarily unstable. The resulting increase in the intracellular concentration of p53 stimulates production of the cyclin-kinase inhibitor p21^{CIP}, which then binds and inhibits the Cdk-cyclin complexes that regulate progress through the cell cycle. As a result, cells with functional p53 and damaged DNA are arrested at the G1 or G2 stage.

The p53 protein can also induce apoptotic mechanisms if DNA damage is too extensive. It has been shown that mutant p53, but not functional wild-type p53, interacts with DAXX, a Fas-binding protein that activates stress-inducible kinase pathways. (Ohiro et al., Mol. Cell. Biol. 23(1): 322-334 (2003)) This interaction inhibits DAXX-dependent activation of apoptosis signal-regulating kinase 1, also known as mitogen-activated protein kinase kinase 5 (MAPKK5). It has been shown that mutant p53 rescued cells from DAXX-dependent inhibition of proliferation.

Mutant p53 is implicated in a number of human cancers, such as breast cancer, colon cancer, tumors of the head and neck, hepatocellular carcinoma, lung cancer, and thyroid cancer. It would be desirable to develop methods for mitigating the downstream effects of the mutant protein, for example by identifying downstream effector proteins and altering the gene expression pattern thereof to drive damaged cells toward nonproliferation or apoptosis.

### SUMMARY OF THE INVENTION

Pharmaceutical compositions are provided that are useful in preventing and treating various cancers. The compositions comprise a pharmaceutically acceptable carrier, and an effective therapeutic or prophylactic amount of an agent that changes the expression pattern of one or more genes related to the cancers. Methods are also provided for the use of the pharmaceutical compositions in the alteration of intracellular levels of cancer-related proteins. In a preferred embodiment, the agent is a nitroxide antioxidant, such as Tempol (4-hydroxy-2,2,6,6-tetramethylpiperidine-1-oxyl), and the cancer is esophageal cancer, hepatocellular carcinoma, colon cancer, prostate cancer, lung cancer, gastric carcinoma, renal cell carcinoma, bone cancer, breast cancer, cervical cancer, brain cancer, or a cancer associated with the tumor suppressor gene p53.

### DETAILED DESCRIPTION OF THE PREFERRED EMBODIMENT

As described above, a composition and method are disclosed which are useful in treating or preventing various cancers. In a preferred embodiment, the agent used to alter expression of genes related to cancer is a nitroxide antioxidant. Tempol is a stable nitroxide radical characterized by the chemical formula 4-hydroxy-2,2,6,6-tetramethylpiperidine-1-oxyl that has antioxidative properties. The present applicants have discovered that Tempol also possesses the novel property of altering the expression of genes encoding for proteins associated with the development or progression of certain cancers (see Table 1 below). Previous therapies have generally not focused on altering the expression patterns of these cancer-related genes.

Tempol accordingly exhibits a novel and unique therapeutic bimodality in such diseases: not only does it directly reduce oxidative stress by eliminating free radicals, but by altering the expression of cancer-associated genes, it also affects the upstream source of several implicated proteins.

The use of other nitroxide compounds is also contemplated. According to certain embodiments the nitroxide compound can be selected from the following formulas:

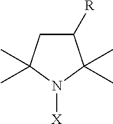

Wherein X is selected from O. and OH, and R is selected from COOH, CONH, CN, and CH₂NH₂.

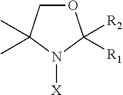

Wherein X is selected from O. and OH, and R₁ is selected from CH₃ and spirocyclohexyl, and R₂ is selected from C₂H₅ and spirocyclohexyl.

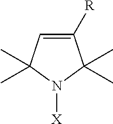

Wherein X is selected from O. and OH and R is selected from CONH.

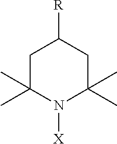

Wherein X is selected from O. and OH and R is selected from H, OH, and NH₂.

Suitable nitroxide compounds can also be found in Proctor, U.S. Pat. No. 5,352,442, and Mitchell et al., U.S. Pat. No. 5,462,946, both of which are hereby incorporated by reference in their entireties.

A non-limiting list of nitroxide compounds include: 2-ethyl-2,5,5-trimethyl-3-oxazolidine-1-oxyl (OXANO), 2,2,6,6-tetramethylpiperidine-1-oxyl (TEMPO), 4-hydroxy-2,2,6,6-tetramethylpiperidine-1-oxyl (TEMPOL), 4-amino-2,2,6,6-tetramethyl-1-piperidinyloxy (Tempamine), 3-Aminomethyl-PROXYL, 3-Cyano-PROXYL, 3-Carbamoyl-PROXYL, 3-Carboxy-PROXYL, and 4-Oxo-TEMPO. TEMPO can also be substituted, typically in the 4 position, for example, 4-amino, 4-(2-bromoacetamido), 4-(ethoxyfluorophosphonyloxy), 4-hydroxy, 4-(2-iodoacetamido), 4-isothiocyanato, 4-maleimido, 4-(4-nitrobenzoyloxyl), 4-phosphonooxy, and the like.

### Experimental Protocol

To assess the effects of Tempol on gene expression, Tempol was administered to experimental mice at a dose of 5 mg/g of food from 14 months to 31 months after birth. Mice receiving the same food without the addition of Tempol were used as a negative control. At the age of 31 months, the experimental animals were sacrificed and the hearts were surgically removed. The expression of a broad spectrum of genes in the cardiac tissue was assessed using chip-based microarray technology. Such chips are well known in the art and are widely used to assess gene expression. The experimental results showed that genes related to various cancers exhibited an alteration in expression. These genes are shown in Table 1.

TABLE 1
CANCER-RELATED GENES EXIBITING ALTERED EXPRESSION
IN CARDIAC TISSUE AFTER TEMPOL ADMINISTRATION

| | | | | | TEMPOL-treated | | | |
|---|---|---|---|---|---|---|---|---|
| | | Control mice | | | mice | | | |
| ORF | Description | tpc1 | tpc2 | tpc3 | tp51 | tp52 | tp53 | Fold change |

DOWNREGULATED GENES

| | | | | | | | | |
|---|---|---|---|---|---|---|---|---|
| D50411 | Meltrin-alpha | 9 | 5 | 26 | −16 | −4 | −14 | −2.3 |
| | (ADAM12) | | | | | | | |
| L28116 | Peroxisome | 33 | 31 | 55 | 1 | 19 | 28 | −2.3 |
| | Proliferator- | | | | | | | |
| | Activated Receptor-δ | | | | | | | |
| M65270 | Cathepsin B | 10 | 8 | 12 | −12 | 6 | 13 | −2.8 |
| AA144746 | Elongation factor-1-δ | 221 | 192 | 167 | 12 | 115 | 185 | −5.9 |

UPREGULATED GENE

| | | | | | | | | |
|---|---|---|---|---|---|---|---|---|
| AA051632 | Mitogen activated | 69 | 25 | −7 | 41 | 84 | 63 | 2.2 |
| | protein kinase kinase 5 | | | | | | | |

In a further gene expression study, Tempol was administered to experimental mice at a dose of 5 g/kg of diet from 12 months through 15 months. Mice receiving the same diet without the addition of Tempol were used as a negative control. At the age of 15 months, the adipose tissue of the experimental animals was obtained. The expression of a broad spectrum of genes in the adipose tissue was assessed using chip-based microarray technology. Specifically, in this case an Affymetrix MOE430A 2.0 array, containing 12,960 genes, was employed. Such chips are well known in the art and are widely used to assess gene expression. The experimental results on the adipose tissue show that genes related to various cancers, exhibited significantly altered expression. These genes are shown in Table 2.

TABLE 2
CANCER-RELATED GENES EXHIBITING INCREASED
EXPRESSION IN ADIPOSE TISSUE AFTER
TEMPOL ADMINISTRATION

| | | | | |
|---|---|---|---|---|
| | Mean (Control | Mean (Tempol- | P | Fold |
| Description | mice) | treated mice) | Value | change |

DOWNREGULATED GENES

| | | | | |
|---|---|---|---|---|
| Hypoxia-inducible | 2727 | 2203 | 0.004 | −1.23 |
| factor, alpha | | | | |
| subunit | | | | |
| (HIF1A) | | | | |
| Superoxide | 5918 | 4423 | 0.004 | −1.33 |
| dismutase 2 | | | | |
| (SOD2) | | | | |
| Uncoupling protein | 3433 | 2140 | 0.012 | −1.61 |
| 2 | | | | |
| (UCP2) | | | | |
| Cyclooxygenase 1 | 392 | 331 | 0.006 | −1.18 |
| (COX-1) | | | | |

UPREGULATED GENES

| | | | | |
|---|---|---|---|---|
| Glutathione S- | 10 | 74 | 0.001 | 7.52 |
| transferase M3 | | | | |
| (GSTM3) | | | | |
| p53-related | 49 | 82 | 0.049 | 1.66 |
| apoptosis effector | | | | |
| related to PMP-22 | | | | |
| (PERP) | | | | |
| Sirtuin 2 | 592 | 686 | 0.048 | 1.16 |
| (SIRT2) | | | | |
| Brain protein I3 | 1549 | 1835 | 0.003 | 1.18 |
| (BRI3) | | | | |

A short summary of the genes described in Tables 1 and 2, and associated cancers, is provided below.

### Genes Exhibiting Altered Expression and Associated Cancer

### 1. Meltrin-Alpha (ADAM12)

The ADAM family of disintegrin and metalloproteinase-containing glycoproteins is highly homologous to the class III snake venom metalloprotease-disintegrins, and has been found to be involved in a variety of cellular processes including sperm-egg interaction, myocyte fusion, neurogenesis, and adipogenesis. Since metalloproteases are known to facilitate malignant phenotype breakdown of the extracellular matrix and cell evasion, a recent study examined ADAM12 in liver tissue specimens from 35 patients with various clinical disorders including cirrhosis, hepatocellular carcinoma, colorectal metastatic disease, nodular hyperplasia, liver donors with extant pathology, and other hepatic metastatic disease. (LePabic et al., Hepatology 37:1056-66 (2003).) ADAM12 mRNA levels were nearly undectectable in both normal livers and those with benign tumors, but were increased 3- to 6-fold in hepatocellular carcinoma and 40- to 60-fold in the livers of patients with metastatic colon cancer, in concert with an increase in matrix metalloproteinase 2 expression and activity. The study concluded that increased ADAM12 expression in liver cancer is associated with tumor aggression and progression.

As shown in Table 1, the expression of ADAM12 in the cardiac tissue of the experimental mice was reduced 2.3-fold in the animals treated with Tempol.

### 2. Peroxisome Proliferator-Activated Receptor-δ

Peroxisome proliferator-activated receptor-δ (PPAR-δ) is one of the PPAR proteins, which are ligand-activated transcription factors that belong to the nuclear hormone receptor superfamily, with PPAR-δ/β involved in embryo implantation and development, epidermal maturation and wound healing, regulation of fatty acid metabolism, repression of the atherogeneric inflammatory response, and perhaps colorectal cancer, where the PPAR-δ gene is overexpressed. In a recent study, APC^{min} mice, which are predisposed to intestinal polyposis, were treated with a selective synthetic agonist of PPAR-δ, resulting in a significant increase in the number and size of intestinal polyps. (Gupta et al., Nature Med. 10:245-47 (2004).) Lesions greater than 2 millimeters in size were increased fivefold in animals given PPAR-δ activator, implicating this agent in the regulation of intestinal adenoma growth.

As shown in Table 1, the expression of PPAR-δ in the cardiac tissue of the experimental mice was reduced 2.3-fold in the animals treated with Tempol.

### 3. Elongation Factor-1-δ

Elongation factor-1 delta (EF-1-δ) is a subunit of EF-1, which is a protein complex that participates in the elongation step of oncogenic transformation, and has recently been considered to be associated with oncogenic transformation. A recent study of cancerous and noncancerous tissue from 52 esophageal carcinoma patients (including both squamous cell carcinoma and adenocarcinoma) undergoing curative esophagectomy found that EF-1-δ was significantly overexpressed in cancerous tissue, with overexpression correlated with both lymph node metastasis and advanced disease staging. (Ogawa et al., British J. Cancer (2004) 91: 282-286.) Moreover, cause-specific survival of patients with higher EF-1-δ expression was significantly poorer than for those with lower expression (5-year survival: 23% vs. 54%, p<0.05).

As shown in Table 1, the expression of EF-1-δ in the cardiac tissue of the experimental mice was reduced 5.9-fold in the animals treated with Tempol.

### 4. Cathepsin B

Cathepsin B is a papain-family cysteine protease that is located in lysosomes where it is involved in protein turnover and maintenance of normal cellular metabolism. Increased expression of the gene encoding for cathepsin B, resulting in a corresponding increase in levels of this enzyme, is observed in several cancers, such as brain cancer, colorectal cancer, lung cancer, and prostate cancer (Yan et al., Biol. Chem. 384:845-54 (2003)). Downregulation of cathepsin B may, therefore, be useful in preventing or treating these cancers.

As shown in Table 1, the expression of cathepsin B in the cardiac tissue of the experimental mice was reduced 2.8-fold in the animals treated with Tempol.

### 5. Mitogen Activated Protein Kinase Kinase 5 (MAPKK5)

As described above, MAPKK5 is capable of interacting with the nuclear protein DAXX. This interaction activates apoptotic pathways such as the JNK cascade. (Ohiro et al., Mol. Cell. Biol. 23(1): 322-334 (2003).) Because mutant p53 itself interacts with DAXX and inhibits activation of MAPKK5, increasing intracellular concentrations of MAPKK5, so as to promote DAXX interaction with MAPKK5 rather than with mutant p53, would be expected to promote activation of the downstream apoptotic cascade and increase the likelihood of destruction of tumor cells with mutant p53.

As shown in Table 2, the expression of MAPKK5 in the cardiac tissue of the experimental mice was increased 2.2-fold in the animals treated with Tempol.

### 6. Glutathione S-Transferase M3 (GSTM3)

The glutathione S-transferases are a large group of dimeric enzymes that are involved in the detoxification of potentially genotoxic electrophilic compounds. The GSTs are phase II metabolic enzymes which catalyze the conjugation of reduced glutathione with potential genotoxic substances, especially those from tobacco smoke. Recent studies have demonstrated an increased risk of lung cancer in individuals having a genotype which is deficient in the GSTM3 enzyme in the lung (Reszka et al., International Journal of Occupational Medicine and Environmental Health 14:2 (2001) 99-113; Mohr et al., Anti-Cancer Research 23 (2003) 2111-2124). Up regulation of GSTM3 may, therefore, be useful in treating or preventing the occurrence of lung cancer.

As shown in Table 2, the expression of GSTM3 was increased 7.52-fold in the adipose tissue of the animals treated with Tempol.

### 7. p53 Related Apoptosis Effector Related to PMP-22 (PERP)

PERP is expressed in a p53-dependent manner and at high levels in apoptotic cells, when compared with G₁ arrested cell (Attardi et al., Genes and Development 14 (2000) 704-718). PERP is a direct p53 target, and its overexpression is sufficient to induce cell death in fibroblasts, showing that it is an important component of p53 apoptotic function. A recent study has demonstrated the involvement of PERP in p53 mediated cell death in certain types of cells, which led the authors to conclude that PERP had a cell type specific role in the p53 cell death pathway (Ihrie et al., Current Biology 13 (2003) 1985-1990). An increase in the intracellular level of the PERP protein in tumor cells might therefore be expected to lead to an increase in apoptosis in tumor cells overexpressing the protein.

As shown in Table 2, the expression of PERP in the adipose tissue of the experimental mice was increased 1.66-fold in the animals treated with Tempol.

### 8. Brain Protein I3 (BRI3)

BRI3 is a brain specific type II membrane protein localized to lysosomes that has been shown to be involved with TNF-induced cell death (Wu et al., Biochemical and Biophysical Research Communications 311 (2003) 518-524). Furthermore, TNF is known to mediate apoptosis in susceptible tumor cell lines. Therefore, an increase in BRI3 expression may be useful in the treatment of tumors in which TNF plays a role in the mediation of apoptosis.

As shown in Table 2, the expression of BRI3 in the adipose tissue of the experimental mice was increased 1.18-fold in the animals treated with Tempol.

### 9. Hypoxia Inducible Factor 1, Alpha Subunit (HIF1A)

HIF1 is a heterodimer composed of an α and a β subunit that mediates the cellular response to reduced oxygen tension or hypoxia. There is strong evidence that indicates that HIF1 contributes to tumor progression. HIF1 controls the expression of gene products that stimulate angiogenesis, such as vascular endothelial growth factor, and that promote metabolic adaptation to hypoxia, such as glucose transporters and glycolytic enzymes. In mouse xenograft models, tumor growth and angiogenesis have been shown to be inhibited by loss of HIF1 activity, and stimulated by the overexpression of HIF1A. HIF1A has been shown to be overexpressed in, among other cancers, esophageal carcinoma (Kurokawa et al., British Journal of Cancer 89 (2003) 1042-1047), gastric carcinoma (Huang et al., Journal of Biomedical Science 12 (2005) 229-241), and renal cell carcinoma (Lidgren et al., Clinical Cancer Research 11 (2005) 1129-1135). The level of expression of HIF1A has been shown to be correlated with tumor grade, angiogenesis, and mortality. Downregulation of HIF1A, may, therefore, be useful in preventing, or retarding the progression of, various types of cancer.

As shown in Table 2, the expression of HIF1 alpha in the adipose tissue of the experimental mice was reduced 1.23-fold in the animals treated with Tempol.

### 10. Superoxide Dismutase 2 (SOD2)

SOD2 is a member of the ion/manganese super oxide dismutase family that codes for enzymes involved in the dismutation or conversion of superoxide into hydrogen peroxide and diatomic oxygen. Recent studies have shown that overexpression of superoxide dismutase in Ewing's carcinoma cells protects the cells against TNF-alpha-induced apoptosis (Djavaheri-Mergny et al., FEBS Letters 578 (2004) 111-115), that overexpression of superoxide dismutase can promote the survival of prostate cancer cells exposed to hypothermic insult (Venkataraman et al., Free Radical Research 38 (10) (2004) 1119-1132), and that an osteocarcinoma cell line overexpressing superoxide dismutase acquired resistance to adriamycin, a common chemotherapeutic agent (Wang et al., International Journal of Oncology 26 (2005) 1291-1300). Furthermore, the expression of superoxide dismutase has been shown in an immunohistochemical study to increase in a direct relationship with tumor grade in invasive breast carcinoma (Tsanou et al., Histology and Histopathology 19 (2004) 807-813). Because superoxide dismutase has been implicated in protecting tumor cells against apoptotic insults, a reduction in the expression of superoxide dismutase would be expected to lead to an increase in the proportion of tumor cells entering apoptosis as a result of targeted therapeutic apoptotic insults.

As shown in Table 2, the expression of super oxide dismutase 2 in the adipose tissue of the experimental mice was reduced 1.33-fold in the animals treated with Tempol.

### 11. Uncoupling Protein 2 (UCP2)

UCP2 is a recently identified mitochondrial inner membrane anion carrier, which has been shown to be a negative regulator of reactive oxygen species production. Such proteins separate oxidative phosphorylation from ATP synthesis with energy dissipated as heat. A recent study provided evidence for the hypothesis that increased expression of UCP2 is one of the adaptive mechanisms that result from oxidative stress in cancer cells: in the study, it was shown that UCP2 expression was increased in human colon cancer in such a way as to correlate with the degree of neoplastic change (Horimoto et al., Clinical Cancer Research 10 (2004) 6203-6207). Because an upregulation in UCP2 is an adaptive mechanism employed by tumor cells to protect against reactive oxygen species, down regulation of UCP2 in the cells would be expected to have a beneficial effect by subjecting the tumor cells to a higher level of oxidative stress.

As shown in Table 2, the expression of uncoupling protein 2 in the adipose tissue of the experimental mice was reduced 1.61-fold in the animals treated with Tempol.

### 12. Cyclooxygenase 1 (COX-1)

Cyclooxygenase 1 (COX-1) (also known as prostaglandin-endoperoxide synthase 1) is a member of a family of enzymes involved in prostaglandin synthesis. The increased expression of COX-1 has been demonstrated in non-small cell lung carcinoma (Hastürk et al., Cancer 94 (4) (2002) 1023-1031; Yoshimoto et al., Oncology Reports 13 (2005) 1049-1057), and an upregulation in COX-1 has also been reported in cervical carcinoma (Sales et al., Cancer Research 62 (2002) 424-432). An elevated level of COX-1 has been shown to be associated in cervical carcinoma with increased expression of various growth factors related to angiogenesis. Therefore, a reduction in the expression level of COX-1 in tumor cells would be expected to have a beneficial effect on the progression of the tumors.

As shown in Table 2, the expression of COX-1 in the adipose tissue of the experimental mice was reduced 1.18-fold in the animals treated with Tempol.

### 13. Sirtuin 2 (SIRT2)

Sirtuin 2 is a cytoskeleton-related protein which has been observed to be downregulated in gliomas (Hiratsuka et al., Biochemical and Biophysical Research Communications 309 (2003) 558-566). It has been postulated that SIRT2 may act as a tumor suppressor in glioma cells. Therefore, an increase in the level of SIRT2 protein in glioma cells would be expected to have a beneficial effect on the progress of such tumors.

As shown in Table 2, the expression of SIRT2 in the adipose tissue of the experimental mice was increased 1.16-fold in the animals treated with Tempol.

### Preferred Embodiment: Cancer Prophylaxis and Treatment Protocol

As described above, Tempol has the effect of altering the expression of genes related to certain cancers. Since the expression of these genes is altered, administration of Tempol will have a beneficial effect by altering concentrations of gene products that are linked to the development or progression of the associated cancers. In a preferred embodiment of the present invention, therefore, Tempol is administered to a mammalian host, such as a human, exhibiting no symptoms of a gene-associated cancer in order to prevent the development of that cancer. Particularly preferred patients are those who are predisposed or otherwise at risk for the cancer, such as those with a family history of the cancer or those with genetic or serum markers associated with the cancer. Alternatively, Tempol may be administered to a human exhibiting symptoms of the cancer or other evidence of cancer initiation or progression, in order to retard or arrest the progress of the cancer. For this purpose, Tempol, non-toxic salts thereof, acid addition salts thereof or hydrates thereof may be administered systemically or locally, usually by oral or parenteral administration.

The doses to be administered are determined depending upon, for example, age, body weight, symptom, the desired therapeutic effect, the route of administration, and the duration of the treatment. In the human adult, the dose per person at a time is generally from about 0.01 to about 1000 mg, by oral administration, up to several times per day. Specific examples of particular amounts contemplated via oral administration include about 0.02, 0.03, 0.04, 0.05, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, 1000 or more mg. The dose per person at a time is generally from about 0.01 to about 300 mg/kg via parenteral administration (preferably intravenous administration), from one to four or more times per day. Specific examples of particular amounts contemplated include about 0.02, 0.03, 0.04, 0.05, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300 or more mg/kg. Continuous intravenous administration is also contemplated for from 1 to 24 hours per day to achieve a target concentration from about 0.01 mg/L to about 100 mg/L. Specific examples of particular amounts contemplated via this route include about 0.02, 0.03, 0.04, 0.05, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more mg/L. The dose to be used does, however, depend upon various conditions, and there may be cases wherein doses lower than or greater than the ranges specified above are used.

Tempol may be administered in the form of, for example, solid compositions, liquid compositions or other compositions for oral administration, injections, liniments or suppositories for parenteral administration.

Solid compositions for oral administration include compressed tablets, pills, capsules, dispersible powders and granules. Capsules include hard capsules and soft capsules. In such solid compositions, Tempol may be admixed with an excipient (e.g. lactose, mannitol, glucose, microcrystalline cellulose, starch), combining agents (hydroxypropyl cellulose, polyvinyl pyrrolidone or magnesium metasilicate aluminate), disintegrating agents (e.g. cellulose calcium glycolate), lubricating agents (e.g. magnesium stearate), stabilizing agents, agents to assist dissolution (e.g. glutamic acid or aspartic acid), or the like. The agents may, if desired, be coated with coating agents (e.g. sugar, gelatin, hydroxypropyl cellulose or hydroxypropylmethyl cellulose phthalate), or be coated with two or more films. Further, coating may include containment within capsules of absorbable materials such as gelatin.

Liquid compositions for oral administration include pharmaceutically acceptable solutions, suspensions, emulsions, syrups and elixirs. In such compositions, Tempol is dissolved, suspended or emulsified in a commonly used diluent (e.g. purified water, ethanol or mixture thereof). Furthermore, such liquid compositions may also comprise wetting agents or suspending agents, emulsifying agents, sweetening agents, flavoring agents, perfuming agents, preserving agents, buffer agents, or the like.

Injections for parenteral administration include solutions, suspensions, emulsions and solids which are dissolved or suspended. In injections, Tempol may be dissolved, suspended and emulsified in a solvent. The solvents are, for example, distilled water for injection, physiological salt solution, vegetable oil, propylene glycol, polyethylene glycol, alcohol such as ethanol, or a mixture thereof. Moreover the injections may also include stabilizing agents, agents to assist dissolution (e.g. glutamic acid, aspartic acid or POLYSORBATE80 (registered trade mark)), suspending agents, emulsifying agents, soothing agents, buffer agents, preserving agents, etc. They are sterilized in the final process or manufactured and prepared by sterile procedure. They may also be manufactured in the form of sterile solid compositions, such as a freeze-dried composition, and they may be sterilized or dissolved immediately before use in sterile distilled water for injection or some other solvent.

Other compositions for parenteral administration include liquids for external use, and ointment, endermic liniments, inhale, spray, suppositories for rectal administration and pessaries for vaginal administration which comprise Tempol and are administered by methods known in the art.

Spray compositions may comprise additional substances other than diluents: e.g. stabilizing agents (e.g. sodium sulfite hydride), isotonic buffers (e.g. sodium chloride, sodium citrate or citric acid). For preparation of such spray compositions, for example, the method described in U.S. Pat. No. 2,868,691 or No. 3,095,355 may be used. Briefly, a small aerosol particle size useful for effective distribution of the medicament may be obtained by employing self-propelling compositions containing the drugs in micronized form dispersed in a propellant composition. Effective dispersion of the finely divided drug particles may be accomplished with the use of very small quantities of a suspending agent, present as a coating on the micronized drug particles. Evaporation of the propellant from the aerosol particles after spraying from the aerosol container leaves finely divided drug particles coated with a fine film of the suspending agent. In the micronized form, the average particle size is less than about 5 microns. The propellant composition may employ, as the suspending agent, a fatty alcohol such as oleyl alcohol. The minimum quantity of suspending agent is approximately 0.1 to 0.2 percent by weight of the total composition. The amount of suspending agent is preferably less than about 4 percent by weight of the total composition to maintain an upper particle size limit of less than 10 microns, and preferably 5 microns. Propellants that may be employed include hydrofluoroalkane propellants and chlorofluorocarbon propellants. Dry powder inhalation may also be employed.

### EXAMPLE 1

A 70-kilogram patient diagnosed with cancer is administered a dose of 1500 mg of Tempol per day for 180 days. This may be administered in a single dose, or may be administered as a number of smaller doses over a 24-hour period: for example, three 500-mg doses at eight-hour intervals. Following treatment, the protein levels of elongation factor-1 delta, ADAM12, cathepsin B, peroxisome proliferator-activated receptor-δ, hypoxia-inducible factor alpha subunit, superoxide dismutase 2, uncoupling protein 2, and cyclooxygenase 1 in the cancerous tissue are reduced, and the levels of mitogen-activated protein kinase kinase 5, glutathione S-transferase M3, PERP, sirtuin 2, and brain protein I3 are increased.

### EXAMPLE 2

A 70-kilogram patient with familial risk factors for cancer but exhibiting no symptoms thereof is administered a dose of 1500 mg of Tempol per day for 180 days. This may be administered in a single dose, or may be administered as a number of smaller doses over a 24-hour period: for example, three 500-mg doses at eight-hour intervals. Following treatment, the protein levels of elongation factor-1 delta, ADAM12, cathepsin B, peroxisome proliferator-activated receptor-δ, hypoxia-inducible factor alpha subunit, superoxide dismutase 2, uncoupling protein 2, and cyclooxygenase 1 in the cancerous tissue are reduced, and the levels of mitogen-activated protein kinase kinase 5, glutathione S-transferase M3, PERP, sirtuin 2, and brain protein I3 are increased.

What is claimed is:

1. A method for altering intracellular levels of one or more proteins associated with a cancer, comprising:
identifying an individual in need of altering levels of cancer-associated proteins; and
administering to that individual an effective amount of a nitroxide antioxidant.

2. The method of claim 1, wherein the nitroxide antioxidant is 4-hydroxy-2,2,6,6-tetramethylpiperidine-1-oxyl.

3. The method of claim 1, where the level of the cancer-associated protein is reduced.

4. The method of claim 3, wherein the cancer-associated proteins are selected from the group consisting of elongation factor-1 delta, ADAM12, cathepsin B, peroxisome proliferator-activated receptor-δ, hypoxia-inducible factor alpha subunit, superoxide dismutase 2, uncoupling protein 2, and cyclooxygenase 1.

5. The method of claim 1, where the level of the cancer-associated protein is increased.

6. The method of claim 5, wherein the cancer-associated proteins are selected from the group consisting of mitogen-activated protein kinase kinase 5, glutathione S-transferase M3, PERP, sirtuin 2, and brain protein I3.

7. The method of claim 1, wherein the cancer is selected from a group consisting of esophageal cancer, hepatocellular carcinoma, colon cancer, prostate cancer, lung cancer, gastric carcinoma, renal cell carcinoma, bone cancer, breast cancer, cervical cancer, brain cancer, and a cancer associated with the tumor suppressor gene p53.

8. The method of claim 1, wherein the effective amount of a nitroxide antioxidant is within a range of 0.01-300 mg/kg.

9. The method of claim 1, wherein the effective amount of a nitroxide antioxidant is within a range of 0.1-250 mg/kg.

10. The method of claim 1, wherein the effective amount of a nitroxide antioxidant is within a range of 1-200 mg/kg.

11. The method of claim 1, wherein the effective amount of a nitroxide antioxidant is within a range of 2-150 mg/kg.

12. The method of claim 1, wherein the effective amount of a nitroxide antioxidant is within a range of 5-125 mg/kg.

13. The method of claim 1, wherein the effective amount of a nitroxide antioxidant is within a range of 7-100 mg/kg.

14. The method of claim 1, wherein the effective amount of a nitroxide antioxidant is within a range of 10-75 mg/kg.

15. The method of claim 1, wherein the effective amount of a nitroxide antioxidant is within a range of 15-30 mg/kg.

16. A method for inhibiting the progression of cancer associated with a protein, comprising:
identifying an individual affected by or at risk for the protein-associated cancer; and
administering to that individual an amount of a nitroxide antioxidant effective to alter expression of a gene associated with the protein-associated cancer.

17. The method of claim 16, where the expression of the gene is reduced.

18. The method of claim 17, wherein the gene is selected from a group consisting of elongation factor-1 delta, ADAM12, cathepsin B, peroxisome proliferator-activated receptor-δ, hypoxia-inducible factor alpha subunit, superoxide dismutase 2, uncoupling protein 2, and cyclooxygenase 1.

19. The method of claim 16, where the expression of the gene is increased.

20. The method of claim 19, wherein the gene is selected from the group consisting of mitogen-activated protein kinase kinase 5, glutathione S-transferase M3, PERP, sirtuin 2, and brain protein I3.

21. The method of claim 16, wherein the protein-associated cancer is selected from a group consisting of esophageal cancer, hepatocellular carcinoma, colon cancer, prostate cancer, lung cancer, gastric carcinoma, renal cell carcinoma, bone cancer, breast cancer, cervical cancer, brain cancer, and a cancer associated with the tumor suppressor gene p53.

22. The method of claim 16, wherein the nitroxide antioxidant is 4-hydroxy-2,2,6,6-tetramethylpiperidine-1-oxyl.

23. The method of claim 16, wherein the effective amount of a nitroxide antioxidant is within a range of 0.01-300 mg/kg.

24. The method of claim 16, wherein the effective amount of a nitroxide antioxidant is within a range of 0.1-250 mg/kg.

25. The method of claim 16, wherein the effective amount of a nitroxide antioxidant is within a range of 1-200 mg/kg.

26. The method of claim 16, wherein the effective amount of a nitroxide antioxidant is within a range of 2-150 mg/kg.

27. The method of claim 16, wherein the effective amount of a nitroxide antioxidant is within a range of 5-125 mg/kg.

28. The method of claim 16, wherein the effective amount of a nitroxide antioxidant is within a range of 7-100 mg/kg.

29. The method of claim 16, wherein the effective amount of a nitroxide antioxidant is within a range of 10-75 mg/kg.

30. The method of claim 16, wherein the effective amount of a nitroxide antioxidant is within a range of 15-30 mg/kg.

31. Use of a nitroxide antioxidant in the preparation of a medicament for altering intracellular levels of one or more proteins associated with a cancer.

32. Use of a nitroxide antioxidant in the preparation of a medicament for inhibiting the progression of cancer associated with a protein.
